(19) United States
(12) Patent Application Publication
(10) Pub. No.: US 2015/0004361 A1
(43) Pub. Date: Jan. 1, 2015

(54) ANTIMICROBIAL DEVICES COMPRISING HYPER-CONDUCTIVE AND DIELECTRIC LAYERS

(71) Applicant: Taylor C. Culpepper, Edmond, OK (US)

(72) Inventor: Taylor C. Culpepper, Edmond, OK (US)

(21) Appl. No.: 14/318,566

(22) Filed: Jun. 27, 2014

Related U.S. Application Data
(60) Provisional application No. 61/957,182, filed on Jun. 27, 2013.
(60) Provisional application No. 61/957,601, filed on Jul. 9, 2013.
(60) Provisional application No. 61/957,603, filed on Jul. 9, 2013.
(60) Provisional application No. 61/959,361, filed on Aug. 22, 2013.
(60) Provisional application No. 61/912,013, filed on Dec. 4, 2013.

(51) Int. Cl. A61L 2/03; A61L 31/04; A61L 15/22; A61L 31/02; A61L 26/00; A61L 15/18

(52) U.S. Cl. CPC A61L 2/03; A61L 26/0004; A61L 26/0009; A61L 15/18; A61L 15/22; A61L 31/022; A61L 31/04
USPC 428/138; 428/457; 442/52; 442/6; 428/337; 428/172; 428/336; 607/115; 156/60; 427/58; 427/123; 427/108

(57) ABSTRACT

Antimicrobial devices such as molded components can include surfaces which have a microbial field disruptive hyper-conductive layer covered by a dielectric surface layer, to continuously disinfect said surfaces. Also, the present invention relates to generally antimicrobial dressings and more particularly to dermal dressings and bandages providing antiseptic disinfection, comprising typical modern dressings and bandages stratified in close proximity to microbial field disruptive hyper-conductive elements or alloys which deactivate microbes by disrupting the electric field generated by and used by the microbes, and isolated the wound or surgical site tissue from said conductors with a layer or layers of dielectric film.

### FIELD OF THE INVENTION

The present invention generally relates to antimicrobial devices and antimicrobial articles comprising surfaces having antimicrobial efficacy.

### BACKGROUND

Current research and practices have illustrated the anti-microbial efficacy of articles and devices containing hyper conducting materials such as copper, silver, and their alloys both in solution and in elemental form. Existing theories rely on transfer of conductor ions to explain the mechanism by which these metals deactivate microbes. Accordingly, current understanding is that for compounds such as copper, silver, and their alloys, one requirement is direct contact between the compound and the microbe or between the compound and the fluid which contains the microbe. Regardless of the mechanism, the microbial lethality of these elements are widely accepted and documented over hundreds if not thousands of years. Recent discoveries have shown that the hyper-conductive elements silver, copper, gold, and aluminum all possess this ability. Accordingly, avenues are available to utilize and improve on such antimicrobial activity.

### SUMMARY

There is a need for daily articles and devices to have a disinfected or sterile surface. Such need arises especially for household items, office items, medical devices, and hospital articles, i.e. items that are handled by multiple persons or come in contact with infected persons or animals. It is well known that metal surfaces have a disinfected or sterile surface as pathogens have a short survival rate when they come into contact with the metal. Among those metals, copper, silver, gold, and other noble metals show antimicrobial efficacys as do alloys of such metals, such as bronze, brass, etc.

However, one disadvantage for the use of noble metals in common articles and items is of course the exorbitant costs of these metals making such articles and items economically unfeasible, while metals such as copper have only limited applicability or are categorized to be toxic for certain applications by the Food and Drug Agency (FDA). Accordingly, herein aspects and embodiments are presented that apply the antimicrobial activity of metals with a great reduction or even elimination of the toxic properties.

These embodiments can be used in numerous different applications, some of which are described below. In one aspect, an antimicrobial device includes a hyper-conductive layer and a dielectric layer. The dielectric layer can be adjacent to the hyper-conductive layer. The dielectric layer forms a barrier between the hyper-conductive layer and the environment. The barrier prevents transfer of material, such as molecules, atoms, ions, or electrons, from the hyper-conductive layer across the dielectric barrier. Likewise, the barrier substantially reduces or prevents transfer of material from the environment across the dielectric layer into the hyper-conductive layer. The hyper-conductive layer establishes an electric field which interact across the dielectric layer with the environment. The electric field has an antimictobial effect onto pathogens and microbes, such as bacteria, archaea, protozoa, algae, viruses, fungi, and colonies of bacteria, archaea, protozoa, algae, viruses, and fungi. This antimicrobial efficacy across the dielectric layer is also called a microbial field disruption effect.

In a second aspect, a molded component can comprise a dielectric material. The dielectric material can have a non-planar surface. The molded component can further include a hyper-conductive layer. The hyper-conductive layer can be adjacent to the dielectric material.

In a third aspect, a fluid container includes a surface configured to contact a fluid. The container can include a dielectric material. The container can further include a hyper-conductive layer. The hyper-conductive layer can be adjacent to the dielectric material. In one embodiment, the surface configured to contact a fluid includes a surface of the dielectric layer. In another embodiment the surface configured to contact a fluid includes a surface of the hyper-conductive material. In one embodiment, a fluid container includes a plurality of surfaces configured to contact a fluid. The plurality of surfaces can be formed by a dielectric material. A hyper-conductive layer can be adjacent to the dielectric material. The plurality of surfaces can be formed by sheets, lamellas, unorganized structures, e.g. porous material or sponge-like material, organized structures, e.g. capillary material or honeycomb structures.

In a fourth aspect, a device includes a non-planar surface and a film. The film can be adjacent to the non-planar surface. The film can include a hyper-conductive layer having a first major surface and a second major surface, wherein the second major surface opposite the first major surface. The film can further include a dielectric layer. The dielectric layer can be adjacent to the first major surface of the hyper-conductive layer. Moreover, the dielectric layer can be opposite to the non-planar surface. In one embodiment, the film can have a maximum thickness of not greater than 20 mil.

In a fifth aspect, a method to prepare material surfaces possessing anti-microbial properties can include using a semi-contiguous extreme low resistivity element. The semi-contiguous extreme low resistivity element can be in the form of a coating, film, foil, perforated foil, woven mesh or unwoven mesh. The semi-contiguous extreme low resistivity element includes a highly conductive layer. The highly conductive layer is configured to disrupt in close proximity any electrical field produced by and for microbial species. The semi-contiguous extreme low resistivity element can be physically and electrically isolated from the exterior surface by a layer of dielectric material.

In a sixth aspect, a method to add anti-microbial properties to dressings and bandages includes using a semi-contiguous extreme low resistivity element. The semi-contiguous extreme low resistivity element can be in the form of a coating, film, foil, perforated foil, woven mesh. Or unwoven mesh. The semi-contiguous extreme low resistivity element can provide a highly conductive plane capable of disrupting in close proximity any electrical field produced by and for microbial species.

Likewise, in a seventh aspect, a method to add anti-microbial properties to surgical sutures can include using a contiguous length of low resistivity element in the form of a wire. The wire can be incorporated in a dielectric layer of insulation. Similarely, a method to add anti-microbial properties to catheters can include incorporating a flexible woven mesh of a hyper-conductive element. The flexible woven mesh of a hyper-conductive element can be embedded within a dielectric material. The flexible woven mesh of a hyper-conductive element with the dielectric material can form the catheter.

In yet an eighth aspect, a method to add anti-microbial properties to prostheses and medical implants includes using a semi-contiguous extreme low resistivity element. The semi-contiguous extreme low resistivity element can be in the form of a coating, film, foil, perforated foil, a woven mesh, or unwoven mesh. The semi-contiguous extreme low resistivity element can provide a highly conductive layer capable of disrupting in close proximity any electrical field produced by and for microbial species.

In a ninth aspect, A method to add anti-microbial properties to containers can include using a semi-contiguous extreme low resistivity element. The semi-contiguous extreme low resistivity element can be in the form of a coating, film, foil, perforated foil, woven mesh, or unwoven mesh. The semi-contiguous extreme low resistivity element can provide a highly conductive layer capable of disrupting in close proximity any electrical field produced by and for microbial species.

Antimicrobial devices such as molded components can include surfaces which have a microbial field disruptive hyper-conductive layer covered by a dielectric surface layer, to continuously disinfect said surfaces. Also, the present invention relates to generally antimicrobial dressings and more particularly to dermal dressings and bandages providing antiseptic disinfection, comprising typical modern dressings and bandages stratified in close proximity to microbial field disruptive hyper-conductive elements or alloys which deactivate microbes by disrupting the electric field generated by and used by the microbes, and isolated the wound or surgical site tissue from said conductors with a layer or layers of dielectric film. Moreover, the present invention further relates to generally antimicrobial prostheses and implant components and more particularly to the various prosthesis sockets and implant parts whose surfaces come in contact with tissue and provide continuous antimicrobial disinfection, comprising typical methods of construction but with the addition of a microbial field disruptive hyper-conductive layer covered by a dielectric layer of material suitable for said components that come in contact with tissue. The present invention also relates to generally antimicrobial containers and more particularly to fluid containers such as a water bottles, thermoses or canteens that provide continuous antimicrobial disinfection, comprising typical methods of construction but with the addition of a microbial field disruptive hyper-conductive layer covered by a dielectric surface layer forming the interior surface of said containers.

Microbes such as bacteria and viruses are deactivated by disrupting the electric field generated by and used by the microbes that are in close proximity to a hyper-conductive layer, and isolated from the said conductor with a layer or layers of dielectric film. The various molding processes can provide continuous antimicrobial disinfection, comprising typical methods of construction but with the addition of a microbial field disruptive hyper-conductive layer covered by a isolating dielectric layer of material suitable for said components.

Physical contact between the hyper-conductive layer and the microbe is not required to achieve a bacterial or viral inactivation as the hyper-conductive element disrupts the electric field of the microbe. The electric field of a microbe is generated by across the microbe's membrane or cell wall, where factors such as ion concentration and pH within the microbe differ from ion concentration or pH outside the microbe and such concentration gradient generates an electric potential across the membrane or cell wall, i.e., the microbe's electric field. Disruption of that field can cause the deactivation or destruction of the microbe. Accordingly, if a microbe's electric field is disrupted for a sufficient period, a microbe is essentially killed (bacteria, fungi) or destroyed (virus). Furthermore, as the effect acts upon the microbe's self generated potential field, a dielectric layer between the highly conductive metal layer and the microbes only slightly decreases the anti-microbial efficacy of the metal layer if at all. Accordingly, one can construct a material combination that affords both the anti-microbial disinfection properties of the hyper-conductive metal and an insulating dielectric layer that isolates the conductor from surface contact, atmospheric oxidation, or contact with biological tissue. Since metal contact with tissue is avoided, complications due to metal toxicities and metal poisonings is prevented. The microbial field disruption method does not rely on any ion transfer, reactions of chemicals or other medicinal materials to deactivate the microbe, only the disruption of its electrical field. The microbial field disruption effect is effective on both gram-negative and gram-positive bacteria, on both enveloped and non-enveloped viruses and on fungi. Additionally, since the dielectric layer provides a protective layer for the hyper-conductive element that substantially prevents oxidation or corrosion, there is substantially no decrease in efficacy over time and thus the container's antimicrobial efficacy has essentially an indefinite life-time.

Molded parts produced by processes such as injection molding or compression molding lend themselves to conversion to microbial field disruption by addition of a hyper-conductive layer underneath a thin layer of the molding material which itself is usually a dielectric polymer. An insert of the desired shape of one of the hyper-conductive materials is placed in the mold (or blank in compression molding) and the molding material forms the desired component as required but now has a hyper-conductive layer several thousandths beneath the surface to be protected by the anti-microbial function. Thus, the components will not change in appearance or function but now will deactivate bacterial and viruses on the surface of the component. The microbial field disruption method does not rely on any ion transfer, chemicals, compounds, solutions or other medicinal materials to deactivate the microbe, only the disruption of its electrical field which leads to its inevitable deactivation. The microbial field disruption effect is effective on both gram-negative an gram-positive bacteria, on both enveloped and non-enveloped viruses and on fungi. Additionally, since the dielectric layer provides a protective layer for the hyper-conductive element that prevents oxidation or corrosion, there is substantially no decrease in efficacy over time and thus the container's antimicrobial efficacy has an indefinite life time.

Although any of the hyper-conductive metals can be used, in particular embodiments copper can be used while silver and gold may be restricted for economic reasons. In some embodiments, copper has a higher efficiacy than aluminum or beryllium. All types of medical devices, bandages, hospital and household articles, prosthesis sockets and implants can be fabricated and modified with an antimicrobial laminate comprising the hyper-conductive layer and the dielectric layer to take advantage of the microbial field disruption effect.

Embodiments of the present invention insulates all biological tissue from physical contact with the anti-microbial elements, i.e. the hyper-conductive layer. This eliminates substantially all negative side effects that result from physical contact. Antimicrobial efficacy does not require ion transport, chemicals, compounds, solutions or other medicinal materials. The microbial field disruption effect can act through not only the insulating dielectric layer but also through additional dressing layers needed such as materials for absorption, breath-ability, or non-stick layers as well as some tissue itself. Additionally, since the dielectric layer provides a protective layer for the hyper-conductive element that substantially prevents oxidation or corrosion, there is no decrease in efficacy over time and thus the dressings have an indefinite shelf life. Although any of the hyper-conductive metals can be used, particular embodiments will most likely use copper over silver and gold for economic reasons, and over aluminum or beryllium for higher efficacy. All types of dressings, bandages, wound matrices, sutures, catheters and the like can be fabricated to take advantage of the microbial field disruption effect.

Prostheses' largest risk of infection occur at the interface with tissue, in most cases this is the socket. As most modern prostheses incorporate a soft polymer sleeve in the socket for comfort and efficient fit, it lends itself to conversion to microbial field disruption by addition of a hyper-conductive layer underneath a thin layer of the existing dielectric polymer used in the sleeve. Implants are much more varied and different methods apply to the different types of implants. Secondly, a different problem exist with implants as they are often susceptible to formation of biofilms. Biofilm is a microbial derived sessile community characterized by cells that are irreversibly attached to a substratum or interface to each other, embedded in a matrix of extracellular polymeric substances that they have produced. However, as the microbes are deactivated when they come in close proximity to the hyper-conductive layer the biofilm never has a chance to begin formation. Implants such as catheters, heart valves and suture rings, stents, implantable stimulators, fracture-fixation devices as well as all others can benefit by having the microbe field disruption layers added to their structure. The microbial field disruption method does not rely on any ion transfer, chemicals, compounds, solutions or other medicinal materials to deactivate the microbe, only the disruption of its electrical field which leads to its inevitable deactivation. The microbial field disruption effect is effective on both gram-negative an gram-positive bacteria, on both enveloped and non-enveloped viruses and on fungi. Additionally, since the dielectric layer provides essentially a protective layer for the hyper-conductive element that substantially prevents oxidation or corrosion, there is essentially no decrease in efficacy over time and thus the device's antimicrobial efficacy has an indefinite life-time.

Moreover, one can construct a material combination that affords both the anti-microbial disinfection properties of the hyper-conductive metal and an insulating dielectric layer that isolates the conductor from the contents of a container as well as atmospheric oxidation. The microbial field disruption method does not rely on any ion transfer, chemicals, compounds, solutions or other medicinal materials to deactivate the microbe, only the disruption of its electrical field. For liquid container, the microbial field disruption effect is effective on both gram-negative an gram-positive bacteria, on both enveloped and non-enveloped viruses and on fungi. Additionally, since the dielectric layer essentially provides a protective layer for the hyper-conductive element that substantially prevents oxidation or corrosion, there is no decrease in efficacy over time and thus the container's antimicrobial efficacy has substantially an indefinite life-time.

Although any of the hyper-conductive metals can be used, the preferred embodiments will most likely favor copper over silver and gold for economic reasons, and over aluminum or beryllium for higher efficacy. All manner of devices, components, surfaces and articles can be fabricated to take advantage of the invention.

### BRIEF DESCRIPTION OF THE DRAWINGS

The accompanying drawings, which are incorporated into and form a part of the specification, illustrate one or more embodiments of the present invention and, together with this description, serve to explain the principles of the invention. The drawing merely illustrates a preferred embodiment of the invention and is not to be construed as limiting the scope of the invention.

FIG. 1 is is an isometric view of a typical representative sample item having flat, convex and concave surfaces, internal and external surfaces as well as sharp and rounded edges.

FIG. 2 is a top view of the sample in FIG. 1 showing section line 4-4 used in subsequent figures.

FIG. 3 is a detail view of the top view in FIG. 2.

FIG. 4 is a cross-sectional view of the typical representative sample taken along the section line 4-4 in FIG. 2.

FIG. 5 is an isometric cross-sectional view of the sample of FIG. 1.

FIG. 6 is a detail view of the detail area shown in FIG. 5.

FIG. 7 is a larger detail view of the detail area shown in FIG. 5.

FIG. 8 is an exploded isometric view of the sample in FIG. 1 showing the layers separated.

FIG. 9 is an isometric view of a part containing molded features that incorporates a hyper-conductive layer beneath a layer of dielectric materical.

FIG. 10 is a top view of the component in FIG. 9 showing a cross-section line used in detail FIGS. 11, 12 and 13.

FIG. 11 is a cross-sectional view of the component in FIG. 10.

FIG. 12 is a detail view of the cross-section in FIG. 11.

FIG. 13 is a detail view of the detail shown in FIG. 12 showing the different layers of hyper-conductive and dielectric material.

FIG. 14 is an exploded isometric view of the component if the layers are separated for illustrative purposes.

FIG. 15 is an exploded, perspective view of a microbial field disruptive dressing assembly constructed in accordance with a preferred embodiment of the present invention.

FIG. 16 is a cross sectional view taken from a plane orthogonal to the face of the required microbial field disruption elements.

FIG. 17 is a is an enlarged view of FIG. 16 showing an arrangement of the required microbial field disruption elements.

FIG. 18 is a cross-sectional view taken from a plane orthogonal to the face

FIG. 19 is an enlarged view of FIG. 18 showing a arrangement of the dressing assembly containing both the microbial field disruption elements and typical dressings components.

FIG. 20 is perspective view a an embodiment of a suture incorporating the microbial field disruption elements.

FIG. 21 is a perspective view of an embodiment of a catheter incorporating the required hyper-conductive element in a flexible woven form within the dielectric material of the catheter itself.

FIG. 22 is an isometric view of a typical residual limb positioned for insertion into the socket of a prosthesis.

FIG. 23 is an end view and cross-sectional view of same of the residual limb with prosthesis attached.

FIG. 24 is an end view and cross-sectional view of same of a typical socket and sleeve assembly.

FIG. 25 is an isometric view of the socket illustrating the inside of the socket.

FIG. 26 is an isometric view of a fracture-fixation device.

FIG. 27 is an end view, cross-sectional view and enlarged cross-sectional view of a typical fracture-fixation device shown in FIG. 26.

FIG. 28 is an isometric view of a typical implantable stimulation device with electrode.

FIG. 29 is an perspective, layered cutaway view of a microbial field disruptive container assembly constructed in accordance with one embodiment.

FIG. 30 is an enlarged view of FIG. 29.

### DETAILED DESCRIPTION OF THE PREFERRED EMBODIMENT(S)

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

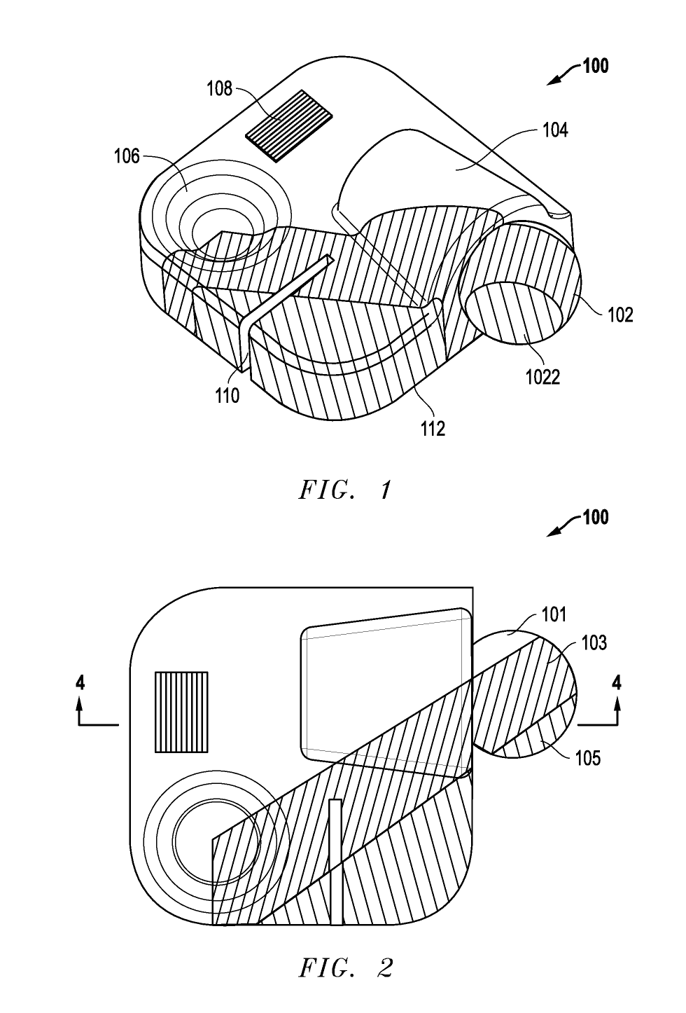

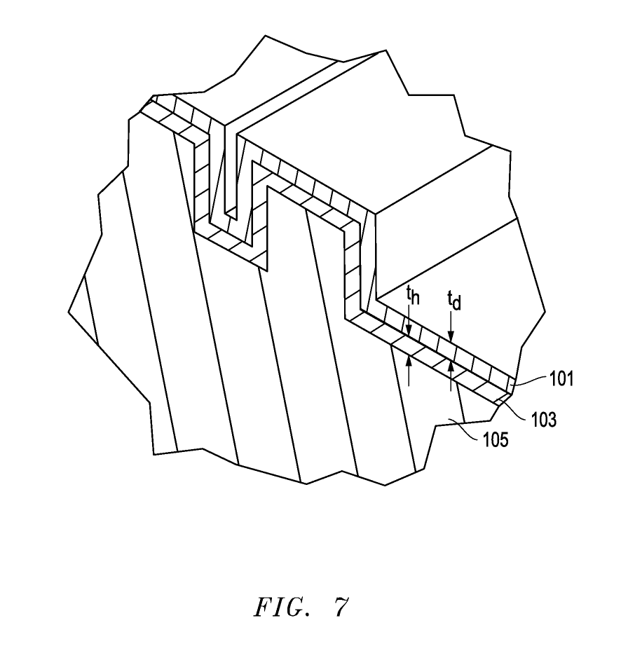

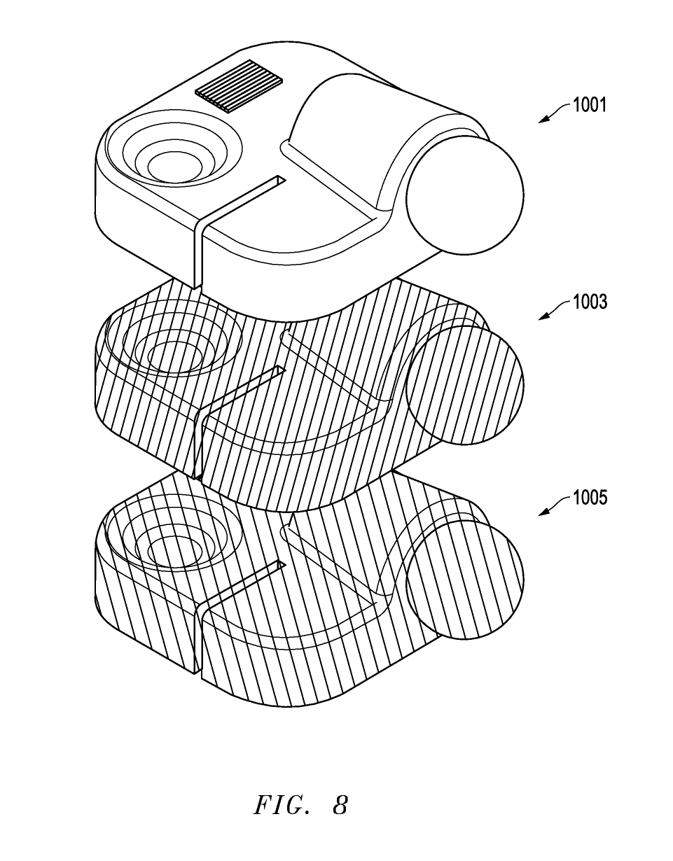

Surfaces FIGS. 1-8

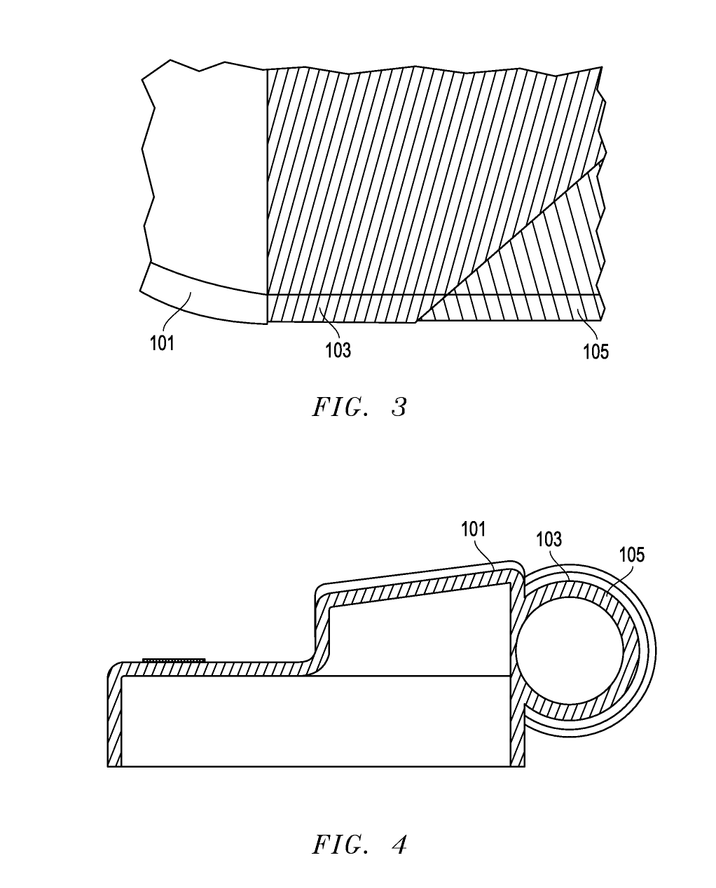

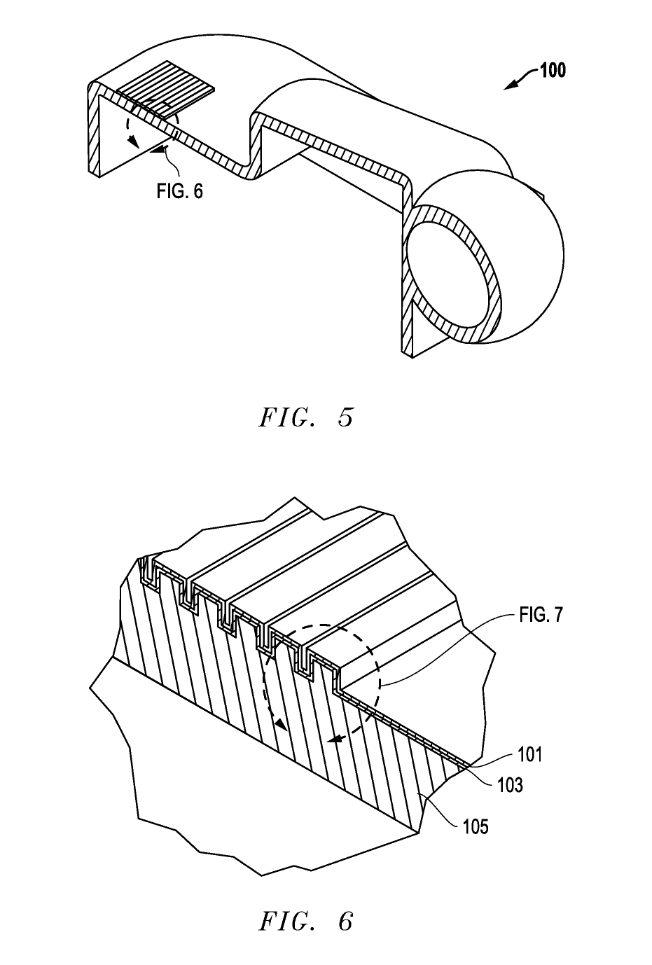

Turning now to the drawings in general and to FIG. 1 in particular, a representative sample of a typical part 100 or component that has flat and both concave (106) and convex (104) curved surfaces, interior and exterior surfaces and both sharp and rounded edges is presented to illustrate that any type of geometry is suitable for being manufactured using the hyper-conductor/dielectric system. The base surface (105) is shown beneath a layer of hyper-conductive layer (103) which is itself beneath a layer of dielectric material (101). Any geometry can have the first hyper-conductive layer (103) and dielectric layer (101) applied as shown on the base elements of a sphere (102), an interior surface (1022) or grooves/channels (108/110). The layering is apparent in FIG. 3 again with the base surface (105), hyper-conductive layer (103) and dielectric layer (101). Additionally a component or assembly can itself be fabricated from the hyper-conductive element itself which would obviously eleminate the need for an additional layer of hyper-conductive material. A cross-section though the the representative sample taken along the section line shown in FIG. 2 is shown in FIG. 3, and an isometric view of same in FIG. 5. Although the layers shown are uniform throughout it is not required. FIG. 6 illustrates a detail of the cross-section with the structure of the base (105) and the hyper-conductive layer (103) conforming to the contours of the base surface (101). While this in and of itself imparts the anti-microbial properties required it is often desired to protect the hyper-conductive material from exposure to corrosion or to prevent the hyper-conductive material from contacting another surface. A polymer or other dielectric material covering the the hyper-conductive material provides this type of protection and allows one to tailor the exterior properties of the component or device. The anti-microbial efficacy of the completed hyper-conductive/dielectric system is only dependent on the the resistivity of the conductors and the both the permittivity and thickness of the dielectric material. The thickness of the hyper-conductive material is only limited to a value where it's resistivity is equivalent to that of the bulk material or about 10 to 20 atomic layers and be of sufficient thickness to form a semi-contiguous, conductive layer capable of disrupting the membrane potential field of microbes. For copper this would theoretically be roughly 2 nm. and research has shown thicknesses less than 50 microns to be effective. Manufacturing practices will more likely use foils that are much more practical to apply such as 0.001″ to 0.005″ thicknesses. The thickness of the dielectric material is dependent upon it's permittivity, the resistivity of the hyper-conductive material beneath it and anti-microbial efficacy desired, and thus the combinations are limitless. Generally high anti-microbial efficacy for copper or silver is achieved within 3 mm for a relative permittivity or dielectric constant of 1.0. The hyper-conductive element (103) may be a coating, foil, perforated foil or woven mesh noting that perforations or voids in a mesh lower the anti-microbial efficacy dependent on their size and ultimate distance to the surface of the dielectric layer. A coating or solid foil facilitates the highest anti-microbial efficacy.

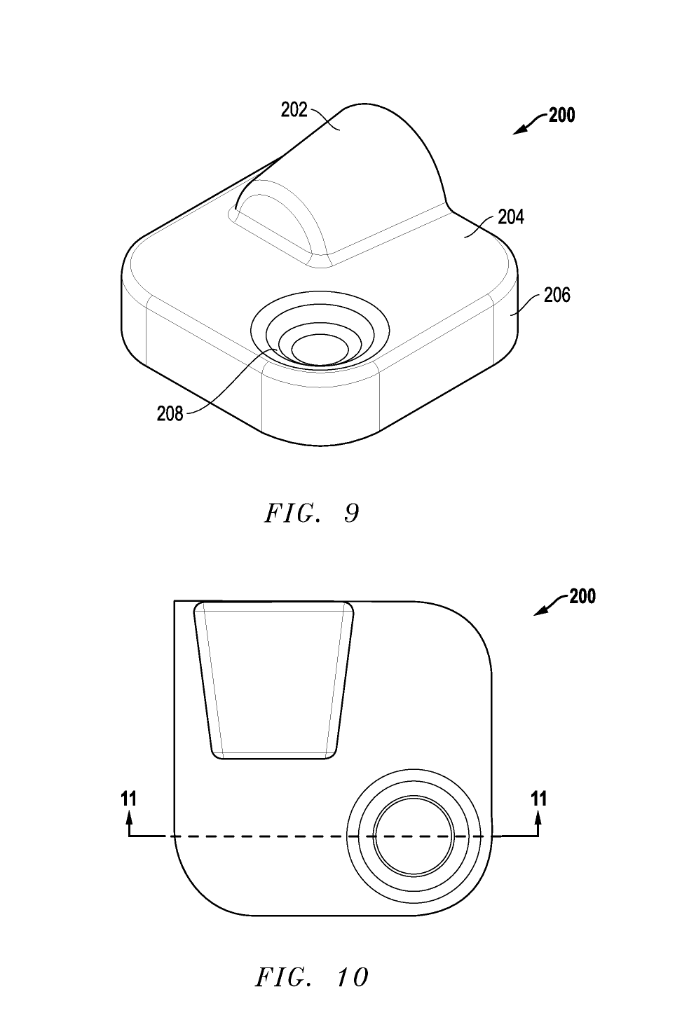

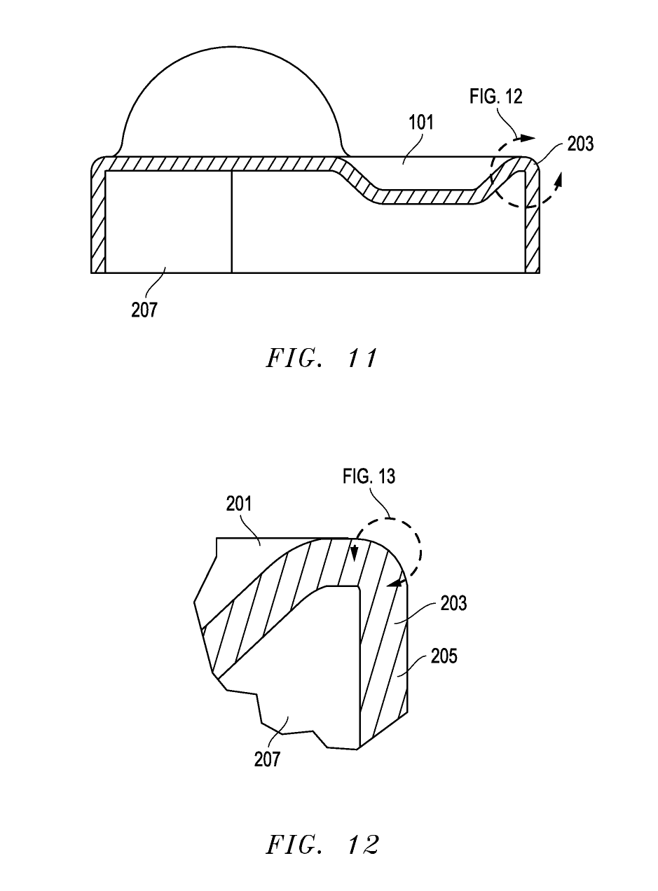

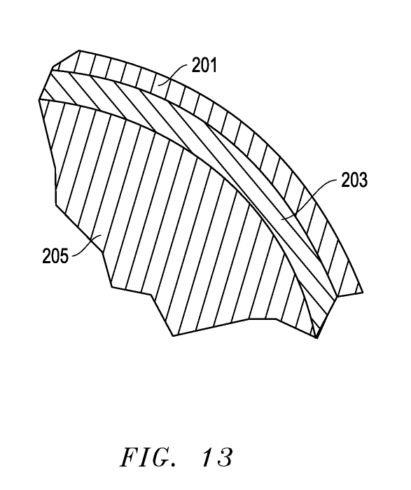

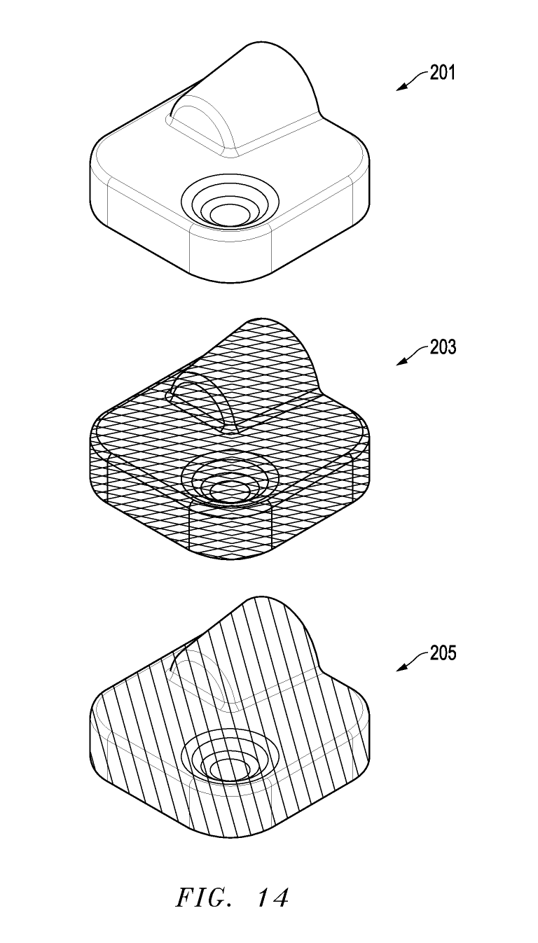

Molded Components FIGS. 9-14

In one embodiment, the hyper-conductive element (103, 203) may be a coating, foil, perforated foil, woven mesh, or unwoven mesh that has less than 40% voids. A coating or solid foil facilitates the highest anti-microbial efficacy. The thickness of the hyper-conductive element depends on which material is used but must be only of sufficient thickness to form a semi-contiguous, conductive layer capable of disrupting the membrane potential field of microbes. Although research has shown thicknesses less than 50 microns to be effective, manufacturing practices will more likely use foils that are much more practical to apply such as 0.001″ to 0.005″ thicknesses. The dielectric layer (101, 201) as well can be well below 0.001″ in thickness, but 0.001″ to 0.003″ films are more practical in mass production and wear longer.

The embodiments shown and described above are exemplary. Many details are often found in the art and, therefore, many such details are neither shown nor described herein. It is not claimed that all of the details, parts, elements, or steps described and shown were invented herein. Even though numerous characteristics and advantages of the present inventions have been described in the drawings and accompanying text, the description is illustrative only. Changes may be made in the details, especially in matters of shape, size, and arrangement of the parts within the principles of the inventions to the full extent indicated by the broad meaning of the terms of the attached claim(s). The description and drawings of the specific embodiments herein do not point out what an infringement of this patent would be, but rather provide an example of how to use and make the invention. Likewise, the abstract is neither intended to define the invention, which is measured by the claims, nor is it intended to be limiting as to the scope of the invention in any way. Rather, the limits of the invention and the bounds of the patent protection are measured by and defined in the following claim(s).

Dressings FIGS. 15-21

Turning to FIG. 15 in particular, a typical modern dressing (300) would comprise an absorptive pad and/or medicinal layer or layers (310) to interface with the wound or surgical site area affixed to a base layer (302) that can also typically have portions with adhesive placed on it. Other layers may include non-stick components or other specific function elements. The current invention introduces the microbial field disruption elements within the typical dressing structure. A contiguous layer of hyper-conductive material such as copper or any of the other extremely low resistivity element or elements or their respective alloys are affixed to a contiguous layer of dielectric film (306) and both are sandwiched within a dressing between the base (302) and functional pad layer or layers (310). As the microbial field disruption effect is an electrical field effect, the proximity of the two layers (304) and (306) to the wound or surgical site area should be kept to a minimum for maximum efficacy. However, the antiseptic benefit does not rely on the additional layers, only the hyper-conductive element (304) and dielectric layer (306) are required. If the the dielectric layer (306) is omitted the antiseptic benefit is still present but unwanted side effects of metal to tissue contact can arise. With this embodiment the wound or surgical site general area is antiseptically protected wherever the hyper-conductive element is in close proximity, but does not have to be in physical contact.

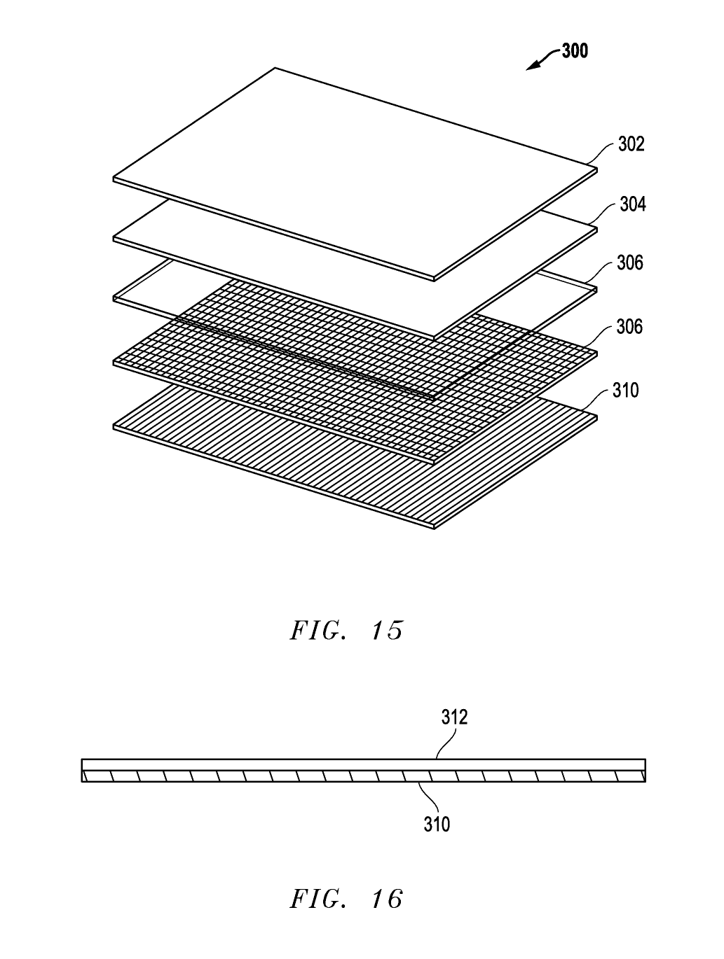

FIG. 16 shows a cross section of the microbial field disruption layer pair(312) and FIG. 17 shows an enlarged view of same. Thus in FIG. 3 the hyper-conductive element is shown affixed to the dielectric layer, and in close proximity to the wound or surgical site area. The hyper-conductive element may be a solid foil, perforated foil or woven mesh that has less than 40% voids. A solid foil facilitates the highest anti-microbial efficacy. The thickness of the hyper-conductive element depends on which material is used but must be only of sufficient thickness to form a semi-contiguous, conductive plane capable of disrupting the membrane potential field of microbes. Although research has shown thicknesses less than 50 microns to be effective, manufacturing practices will more likely use foils that are much more practical to apply such as 0.001″ to 0.005″ thicknesses. The dielectric layer as well can be well below 0.001″ in thickness, but 0.001″ to 0.003″ films are more practical in mass production.

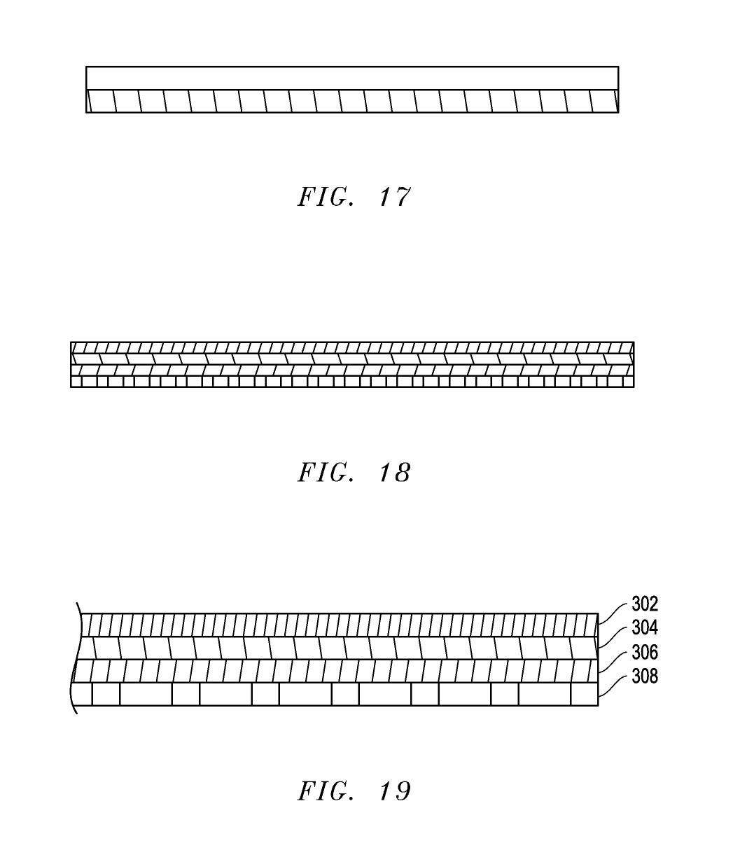

FIG. 18 shows an embodiment wherein the microbe field disruption pair embedded within a typical dressing with base layer and functional pad layers and FIG. 19 is an enlarged view of FIG. 18. Depending on materials chosen the field disruption can extend as far as 0.10″ from the hyper-conductive element (304) closest surface.

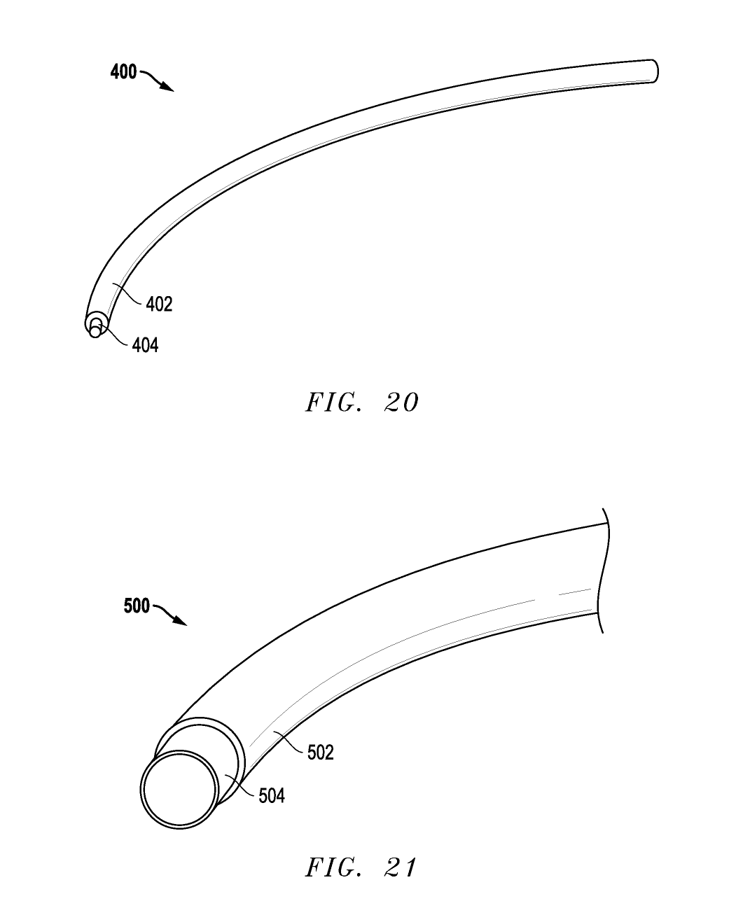

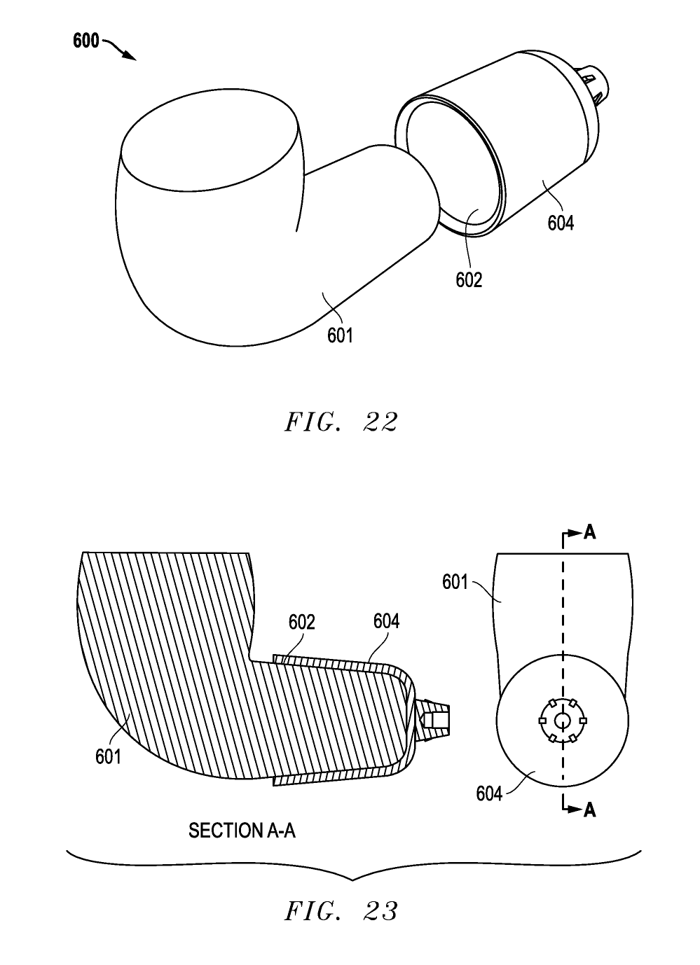

FIG. 20 shows an embodiment of a surgical suture (400) with a hyper-conductive element (404) core and dielectric layer (402), thus rendering the entire length of the suture anti-microbial, and FIG. 21 shows a catheter (500) which serves as the dielectric layer (502) and the hyper-conductive element (504) as a woven mesh several thousandths below the outer surface.

The embodiments shown and described above are exemplary. Many details are often found in the art and, therefore, many such details are neither shown nor described herein. It is not claimed that all of the details, parts, elements, or steps described and shown were invented herein. Even though numerous characteristics and advantages of the present inventions have been described in the drawings and accompanying text, the description is illustrative only. Changes may be made in the details, especially in matters of shape, size, and arrangement of the parts within the principles of the inventions to the full extent indicated by the broad meaning of the terms of the attached claim(s). The description and drawings of the specific embodiments herein do not point out what an infringement of this patent would be, but rather provide an example of how to use and make the invention. Likewise, the abstract is neither intended to define the invention, which is measured by the claims, nor is it intended to be limiting as to the scope of the invention in any way. Rather, the limits of the invention and the bounds of the patent protection are measured by and defined in the following claim(s).

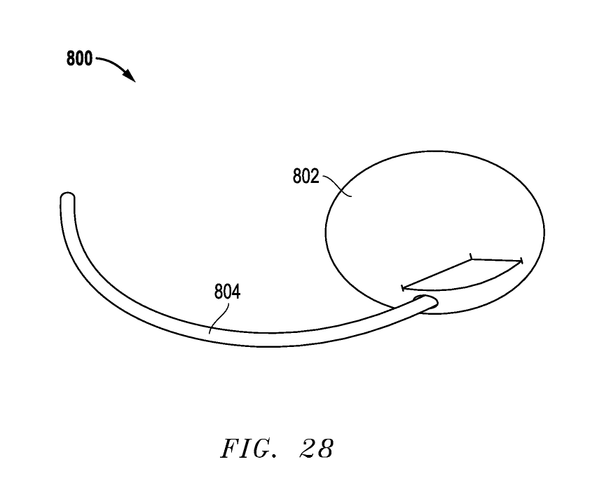

Prostheses FIGS. 22-28

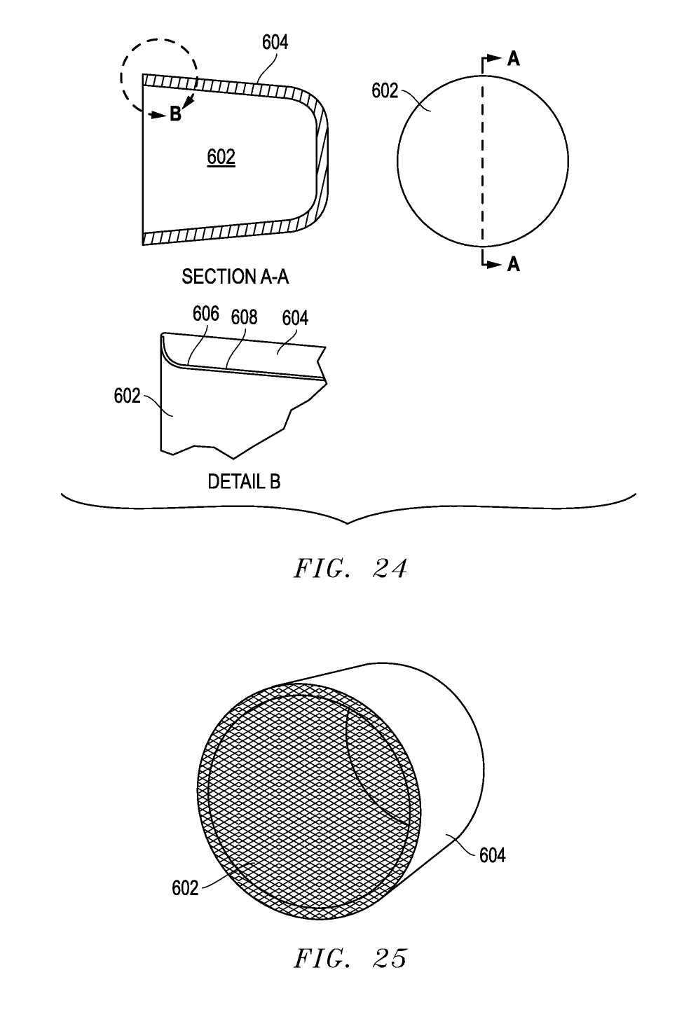

Turning now to FIG. 22 in particular, a typical prosthesis (600) is formed to match the contours of the residual limb (601) for a firm and effective fit. A soft polymer sleeve (602) is typically provided to improve both fit and comfort that fits both the outer contour of the residual limb (601) and the inner contour of the socket (604). The sleeve (602) is shown in cross-section in FIG. 24 where it is comprised of a thin dielectric layer (608) and a hyper-conductive layer (606). Wherever the hyper-conductive layer (606) is present the anti-microbial properties are in effect, so if the hyper-conductive layer is present over the entire area (FIG. 23) where the residual limb (601) contacts the sleeve (602) all microbes with be continually deactivated.

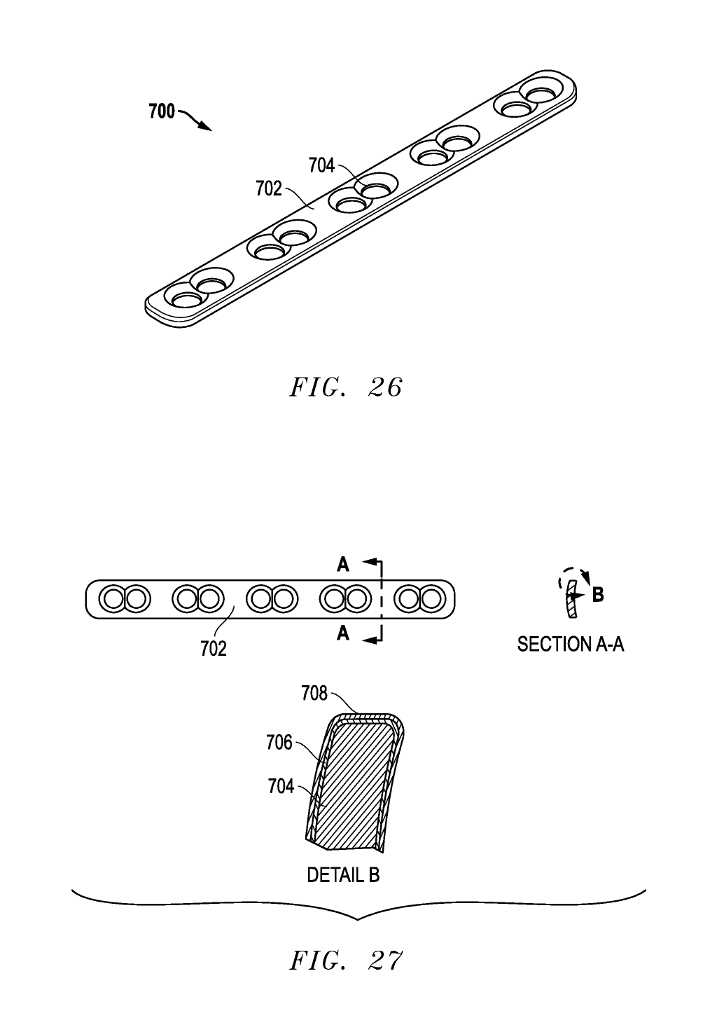

FIG. 26 show a representation of a typical fracture-fixation device (700) used to stabilize a fractured bone. The fixation device (700) can be attached to the bone with fasteners in the holes in the face of the fixation device (700). The device can be temporary of long-term and there are many forms, materials and sizes used. Any of the devices (700) (FIG. 26) or similar implants can incorporate the hyper-conductive layer (706) affixed or coated on the substrate (704). The dielectric layer (708) then covers the hyper-conductive layer (706). Similarly in FIG. 28, a implantable device (800) with electrode(s) (804) incorporate a hyper-conductive layer (802) covered by a dielectric layer (not shown).

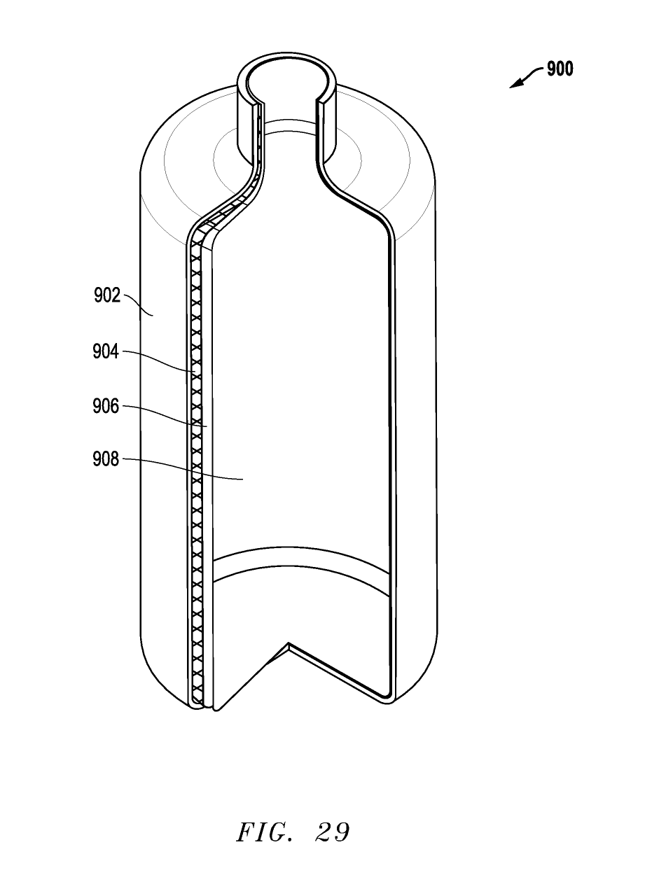

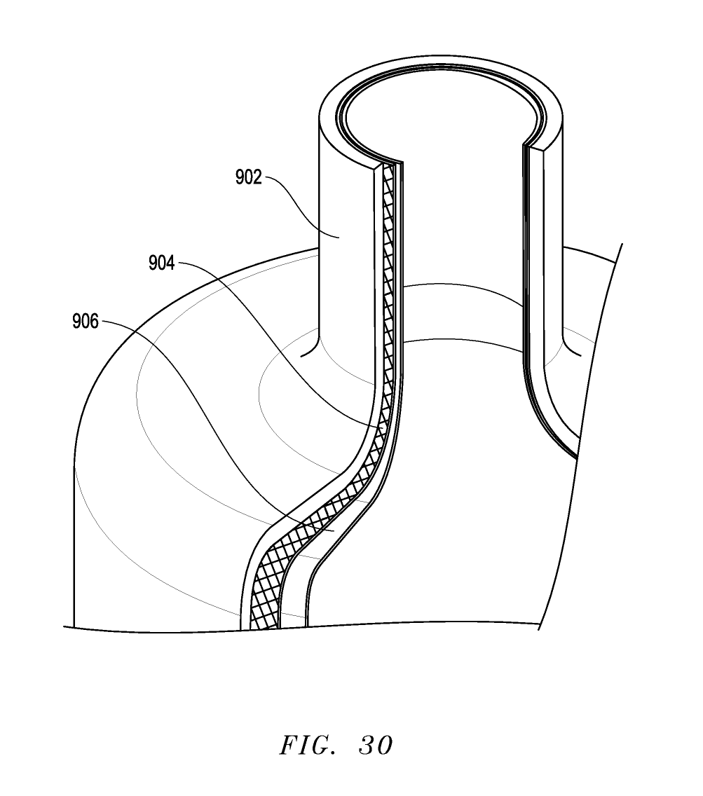

Fluid Containers FIGS. 29-30

Turning now to FIG. 29 in particular, a container (900) would comprise an outer shell (902) of any closed geometric shape capable of holding a fluid with one or more openings for filling/emptying the fluid. The material and thickness of the container are not restricted in any way. On the inner surface of the shell (902) the hyper-conductive element (904) is applied, molded or otherwise deposited preferably covering the entire inner surface. On the inner surface of the hyper-conductive element (904) is a dielectric material (906). The thickness of the dielectric material (906) is sufficient to completely cover the hyper-conductive element (904) but not thick enough to significantly lower the field disruption properties of the hyper-conductive element (904).

In one embodiment, the fluid contained within the container (908) is continuously disinfected as fluid motion brings microbes into close proximity to the hyper-conductive element/dielectric pair structure and deactivates them.

The hyper-conductive element (904) may be a coating, foil, perforated foil or woven mesh that has less than 40% voids. A coating or solid foil facilitates the highest anti-microbial efficacy. The thickness of the hyper-conductive element depends on which material is used but must be only of sufficient thickness to form a semi-contiguous, conductive layer capable of disrupting the membrane potential field of microbes. Although research has shown thicknesses less than 50 microns to be effective, manufacturing practices will more likely use foils that are much more practical to apply such as 0.001″ to 0.005″ thicknesses. The dielectric layer as well can be well below 0.001″ in thickness, but 0.001″ to 0.003″ films are more practical in mass production and wear longer.

The embodiments shown and described above are exemplary. Many details are often found in the art and, therefore, many such details are neither shown nor described herein. It is not claimed that all of the details, parts, elements, or steps described and shown were invented herein. Even though numerous characteristics and advantages of the present inventions have been described in the drawings and accompanying text, the description is illustrative only. Changes may be made in the details, especially in matters of shape, size, and arrangement of the parts within the principles of the inventions to the full extent indicated by the broad meaning of the terms of the attached claim(s). The description and drawings of the specific embodiments herein do not point out what an infringement of this patent would be, but rather provide an example of how to use and make the invention. Likewise, the abstract is neither intended to define the invention, which is measured by the claims, nor is it intended to be limiting as to the scope of the invention in any way. Rather, the limits of the invention and the bounds of the patent protection are measured by and defined in the following claim(s).

The following item list discloses some of the embodiments and properties of the embodiments:

Item 1. An antimicrobial device comprising:
a hyper-conductive layer; and
a dielectric layer adjacent to the hyper-conductive layer, wherein the antimicrobial device, has sufficient antimicrobial efficacy by a microbial field disruption effect.

Item 2. The antimicrobial device according to item 1 comprising an antimicrobial epidermal product or wound care product.

Item 3. A molded component comprising:
a dielectric material having a non-planar surface, and
a hyper-conductive layer adjacent to the dielectric material.

Item 4. The antimicrobial device, the antimicrobial epidermal product or wound care product, or the molded component according to any one of the preceding items, wherein the dielectric layer is in direct contact with the hyper-conductive layer.

Item 5. The antimicrobial device, the antimicrobial epidermal product or wound care product, or the molded component according to any one of the preceding items, wherein the hyper-conductive layer includes a metal.

Item 6. The antimicrobial device, the antimicrobial epidermal product or wound care product, or the molded component according to item 5, wherein the metal includes copper, silver, gold, aluminum, beryllium, or any alloys containing copper, silver, gold, aluminum, beryllium.

Item 7. The antimicrobial device, the antimicrobial epidermal product or wound care product, or the molded component according to any one of the preceding items, wherein the hyper-conductive layer is in a form of a solid foil, a perforated foil, film, a woven mesh, a non-woven mesh, or a grid.

Item 8. The antimicrobial device, the antimicrobial epidermal product or wound care product, or the molded component according to item 7, wherein the woven mesh has less than 40% voids.

Item 9. The antimicrobial device, the antimicrobial epidermal product or wound care product, or the molded component according to any one of the preceding items, wherein the hyper-conductive layer has a thickness sufficient to form a conductive plane.

Item 10. The antimicrobial device, the antimicrobial epidermal product or wound care product, or the molded component according to item 9, wherein the thickness of the hyper-conductive layer is less than 50 microns.

Item 11. The antimicrobial device, the antimicrobial epidermal product or wound care product, or the molded component according to item 9, wherein the thickness of the hyper-conductive layer is between 0.001″ and 0.005″.

Item 12. The antimicrobial device, the antimicrobial epidermal product or wound care product, or the molded component according to any one of the preceding items, wherein the dielectric layer comprises a dielectric polymer.

Item 13. The antimicrobial device, the antimicrobial epidermal product or wound care product, or the molded component according to any one of the preceding items, wherein the dielectric layer has a thickness sufficient to isolate the hyper-conductive layer from direct contact with a microbe.

Item 14. The antimicrobial device, the antimicrobial epidermal product or wound care product, or the molded component according to item 13, wherein the thickness of the dielectric layer is less than 0.001″.

Item 15. The antimicrobial device, the antimicrobial epidermal product or wound care product, or the molded component according to item 13, wherein the thickness of the dielectric layer is in a range from 0.001″ to 0.003″.

Item 16. The antimicrobial device, the antimicrobial epidermal product or wound care product, or the molded component according to any one of the preceding items, wherein the hyper-conductive layer or the dielectric layer can change shape without damaging the hyper-conductive layer or without damaging the dielectric layer.

Item 17. The antimicrobial device, the antimicrobial epidermal product or wound care product, or the molded component according to any one of the preceding items, wherein the antimicrobial device, the antimicrobial epidermal product or wound care product, or the molded component has an antimicrobial efficacy within 3 mm of a surface of the hyper-conductive layer.

Item 18. The antimicrobial device, the antimicrobial epidermal product or wound care product, or the molded component according to any one of the preceding items, further comprising a base layer overlying the hyper-conductive layer, the base layer located opposite to the dielectric layer.

Item 19. The antimicrobial device, the antimicrobial epidermal product or wound care product, or the molded component according to any one of the preceding items, further comprising another dielectric layer adjacent to the hyper-conductive layer and opposite to the dielectric layer.

Item 20. The antimicrobial device, the antimicrobial epidermal product or wound care product, or the molded component according to any one of the preceding items, wherein the antimicrobial device, the antimicrobial epidermal product or wound care product, or the molded component according to any one of the preceding items is an external medical article, an internal medical article, or a non-medical article.

Item 21. The antimicrobial device, the antimicrobial epidermal product or wound care product, or the molded component according to item 20, wherein the external medical article includes a bandage, gauze, compression, tube, triangular, dressings, absorptive, gauze, woven, filler, drape, cover, sterilization wraps, envelope, prosthetic sleeve, plasma bag, IV bag, IV tubing, IV fitting, communication device, button, cord, telephone, pain regulation patient pendant, probe, sphygmomanometer sleeve, sphygmomanometer cuff, tray, container, bed rail, chair, arm rest, or over-bed table surfaces.

Item 22. The antimicrobial device, the antimicrobial epidermal product or wound care product, or the molded component according to item 20, wherein the internal medical article includes an airway device, esophageal device, fixation appliance, balloon, bronchial tube, bone cap, cardiac support mesh, catheter, tube, fixation cerclage, hyberbaric chamber, clamp, implantable clip, vascular clip, traction component, condylar plate fixation implant, blood tubing, blood tubing connector, cover, nerve cuff, neurovascular embolization device, vascular device, bone fusion dowel, pacemaker electrode, spinal epidural electrode, skin expander, cranioplasty plate, cranioplasty plate fastener, internal fibrillator, blood filter, intravascular filter, cardiovascular filter, vascular graft, coronary guidewire, hearing aid, bone conduction, implanted heat-exchanger, cardiopulmonary bypass, dental implant, synthetic implant, radio frequency transponder system, intervertebral fusion device, intracatheter, keratoprosthesis, wire lock, radiographic marker, surgical mesh, ear mold, nail, a pacemaker, a non-invasive pacemaker, patch, pin, bone plate, port, ankle prosthesis, internal chin prosthesis, ear prosthesis, elbow prosthesis, esophageal prosthesis, facial prosthesis, fallopian tube prosthesis, finger prosthesis, hip prosthesis, knee prosthesis, nose prosthesis, otoplasty prosthesis, penile prosthesis, shoulder prosthesis, tendon prosthesis, toe prosthesis, vascular graft prosthesis, wrist prosthesis, retinal prosthesis, blood pump, cardiac event recorder, annuloplasty ring, fixation rod, prosthetic heart valve rotator, bone fixation screw, scleral shell, central nervous system shunt, fixation staple, tibial stent, ureteral stent, peripheral nerve stimulator, spinal-cord stimulator, surgical film, appendage closure system, prostate system rod, surgical tape, oxygen tent, tracheal tube, shunt tube, face mask, surgical washer, or surgical bolt nut.

Item 23. The antimicrobial device, the antimicrobial epidermal product or wound care product, or the molded component according to item 20, wherein the internal non-medical article includes a countertop surface, kitchen surface, bathroom surface, laminated panel, toothbrush, fluid container, food storage container, handle, knob, handle wrap, food preparation pad, food preparation tool, food tray, cup, reception counter, desk pad, pen cup, pen, electrical switches, pacifier, infant toy, crib, carrier, car seat, high chair, high chair tray, keyboard, computer mouse, computer trackball, touchscreen, elevator button, vending machine button, ATM button, telephone button, street crossing button, shopping cart handle, or fuel pump nozzle handle,

Item 24. A fluid container comprising:
a surface configured to contact a fluid; the container comprising
   a dielectric material, and
   a hyper-conductive layer adjacent to the dielectric material; wherein the surface includes a surface of the dielectric layer.

Item 25. A fluid container comprising:
a surface configured to contact a fluid; the container comprising
   a dielectric material, and
   a hyper-conductive layer adjacent to the dielectric material; wherein the surface includes a surface of the hyper-conductive layer.

Item 26. An antimicrobial prosthesis comprising a socket to receive a residual limb, the socket comprising the molded component according to any one of items 1-19, wherein the surface forms an interface between the socket and the residual limb.

Item 27. A device comprising
a non-planar surface and a film adjacent to the non-planar surface, the film comprising:
   a hyper-conductive layer having a first major surface and a second major surface, the second major surface opposite the first major surface; and
   a dielectric layer adjacent to the first major surface of the hyper-conductive layer, the dielectric layer being opposite to the non-planar surface, wherein the film has a maximum thickness of not greater than 20 mil.

Item 28. The device according to item 27, wherein the film consists essentially of the hyper-conductive layer and the dielectric layer.

Item 29. The device according to any one of items 27 or 28, wherein the film has a minimum thickness of at least 0.4 mil, at least 0.8 mil, at least 1.2 mil, at least 1.6 mil, at least 2 mil, at least 2.4 mil, at least 2.8 mil, at least 3.2 mil, at least 3.6 mil, or at least 4 mil.

Item 30. The device according to any one of items 27 through 29, wherein the maximum thickness is not greater than 18 mil, not greater than 16 mil, not greater than 14 mil, not greater than 12 mil, not greater than 10 mil, not greater than 9 mil, not greater than 8 mil, not greater than 7.5 mil, not greater than 7, not greater than 6.5 mil, not greater than 6 mil, not greater than 5.5 mil, not greater than 5 mil, not greater than 4.5 mil, not greater than 4 mil, not greater than 3.5 mil, not greater than 3 mil, not greater than 2.5 mil, not greater than 2 mil, not greater than 1.5 mil, or not greater than 1 mil.

Item 31. The device according to any one of items 27 through 30, wherein the dielectric layer comprises a dielectric material, the dielectric material having a relative permittivity of not greater than 80, not greater than 60, not greater than 40, not greater than 30, not greater than 20, not greater than 15, not greater than 12, not greater than 10, not greater than 9, not greater than 8, not greater than 7, not greater than 6, not greater than 5, not greater than 4.5, not greater than 4, not greater than 3.5, not greater than 3, not greater than 2.8, not greater than 2.6, not greater than 2.4, not greater than 2.2, not greater than 2, or not greater than 1.8.

Item 32. The device according to any one of items 27 through 31, wherein the dielectric layer has a thickness t_{d} and a relative permittivity εᵣ, wherein the product t_{d}*εᵣ is not greater than 500 microns, not greater 450 microns, not greater 400 microns, not greater 350 microns, not greater 300 microns, not greater 250 microns, not greater 200 microns, not greater 150 microns, not greater 140 microns, not greater 130 microns, not greater 120 microns, not greater 110 microns, not greater 100 microns, not greater 90 microns, not greater 80 microns, not greater 70 microns, not greater 60 microns, not greater than 50 microns, not greater than 40 microns, or not greater than 30 microns.

Item 33. The device according to any one of items 27 through 32, wherein the dielectric layer comprises a dielectric material, the dielectric material having a relative permittivity of at least 1.01, at least 1.05, at least 1.08, at least 1.1, at least 1.2, at least 1.3, at least 1.4, or at least 1.5.

Item 34. The device according to any one of items 27 through 33, wherein the dielectric layer comprises a dielectric material, the dielectric material comprising a polymer, a silicon dioxide, a wood pulp product, or any combination thereof.

Item 35. The device according to item 34, wherein the polymer includes a polyolefin, a fluoropolymer, a polyester, polyacrylate, polyurethane, polyimide, polyamide, polyamideimide, polyether, polyketone, polyether ketone, polyether ether ketone, polyphenol formaldehyde, or any combination thereof.

Item 36. The device according to item 35, wherein the polyolefin is selected from high density polyethylene, low density polyethylene, polypropylene, polystyrene, polyvinyltoluene, polyethylene terphthalate, or any combination thereof.

Item 37. The device according to item 35, wherein the fluoropolymer includes a polytetrafluoroethylene (PTFE), polyvinylfluoride (PVF), polyvinylidene fluoride (PVDF), polychlorotrifluoroethylene (PCTFE), perfluoroalkoxy polymer (PFA), fluorinated ethylene-propylene (FEP), polyethylenetetrafluoroethylene (ETFE), polyethylenechlorotrifluoroethylene (ECTFE), or any combination thereof.

Item 38. The device according to item 34, wherein the wood pulp product includes paper, cellulose, cellulose acetate, or any combination thereof.

Item 39. The device according to any one of items 27 through 38, wherein the dielectric layer has an oxygen transmission rate of not greater than 3 cm³/m²/(day*atm), not greater than 2.5 cm³/m²/(day*atm), not greater than 2 cm³/m²/(day*atm), not greater than 1.5 cm³/m²/(day*atm), not greater than 1.4 cm³/m²/(day*atm), not greater than 1.3 cm³/m²/(day*atm), not greater than 1.2 cm³/m²/(day*atm), not greater than 1.1 cm³/m²/(day*atm), not greater than 1.0 cm³/m²/(day*atm), not greater than 0.9 cm³/m²/(day*atm), not greater than 0.8 cm³/m²/(day*atm), not greater than 0.7 cm³/m²/(day*atm), not greater than 0.6 cm³/m²/(day*atm), not greater than 0.5 cm³/m²/(day*atm), not greater than 0.4 cm³/m²/(day*atm), or not greater than 0.3 cm³/m²/(day*atm).

Item 40. The device according to any one of items 27 through 39, wherein the dielectric layer has a carbon dioxide transmission rate of not greater than not greater than 2 cm³/m²/(day*atm), not greater than 1.5 cm³/m²/(day*atm), not greater than 1.4 cm³/m²/(day*atm), not greater than 1.3 cm³/m²/(day*atm), not greater than 1.2 cm³/m²/(day*atm), not greater than 1.1 cm³/m²/(day*atm), not greater than 1.0 cm³/m²/(day*atm), not greater than 0.9 cm³/m²/(day*atm), not greater than 0.8 cm³/m²/(day*atm), not greater than 0.7 cm³/m²/(day*atm), not greater than 0.6 cm³/m²/(day*atm), not greater than 0.5 cm³/m²/(day*atm), not greater than 0.4 cm³/m²/(day*atm), not greater than 0.3 cm³/m²/(day*atm), not greater than 0.2 cm³/m²/(day*atm), or not greater than 0.15 cm³/m²/(day*atm).

Item 41. The device according to any one of items 27 through 40, wherein the dielectric layer has a water vapor transmission rate at 1 atm and 50% humidity of not greater than 3 cm³/m²/day, not greater than 2.5 cm³/m²/day, not greater than 2 cm³/m²/day, not greater than 1.5 cm³/m²/day, not greater than 1.4 cm³/m²/day, not greater than 1.3 cm³/m²/day, not greater than 1.2 cm³/m²/day, not greater than 1.1 cm³/m²/day, not greater than 1.0 cm³/m²/day, not greater than 0.9 cm³/m²/day, not greater than 0.8 cm³/m²/day, not greater than 0.7 cm³/m²/day, not greater than 0.6 cm³/m²/day, not greater than 0.5 cm³/m²/day, not greater than 0.4 cm³/m²/day, or not greater than 0.3 cm³/m²/day.

Item 42. The device according to any one of items 27 through 41, wherein the dielectric layer has a surface roughness Rₐ of not greater than 500 microns, not greater than 100 microns, not greater than 80 microns, not greater than 60 microns, not greater than 50 microns, not greater than 40 microns, not greater than 35 microns, not greater than 30 microns, not greater than 25 microns, not greater than 20 microns, not greater than 15 microns, not greater than 10 microns, or not greater than 5 microns.

Item 43. The device according to any one of items 27 through 42, wherein the dielectric layer has a surface roughness Rₐ of at least 1 micron, at least 4 microns, at least 8 microns, at least 12 microns, at least 16 microns, at least 20 microns, at least 28 microns, or at least 36 microns.

Item 44. The device according to any one of items 27 through 43, wherein the hyper-conductive layer comprises a hyper-conductive material, the hyper-conductive material having an electrical resistivity at 20 deg C. of not greater than 9 μΩ*cm (microohm centimeter), not greater than 8 μΩ*cm, not greater than 7 μΩ*cm, not greater than 6.5 μΩ*cm, not greater than 6 μΩ*cm*m, not greater than 5.5 μΩ*cm, not greater than 5 μΩ*cm, not greater than 4.5 μΩ*cm, not greater than 4 μΩ*cm, not greater than 3.5 μΩ*cm, not greater than 3 μΩ*cm, not greater than 2.8 μΩ*cm, or not greater than 2.6 μΩ*cm, not greater than 2.4 μΩ*cm, not greater than 2.2 μΩ*cm, not greater than 2 μΩ*cm, or not greater than 1.9 μΩ*cm.

Item 45. The device according to any one of items 27 through 44, wherein the hyper-conductive layer comprises a hyper-conductive material including a metal, a non-metallic compound, or any combination thereof.

Item 46. The device according to item 45, wherein the metal includes silver, gold, copper, zinc, aluminum, beryllium, magnesium, lithium, nickel, cobalt, tungsten, titanium, chromium, or an alloy of silver, gold, copper, zinc, aluminum, beryllium, magnesium, lithium, nickel, cobalt, tungsten, titanium, chromium.

Item 47. The device according to item 46, wherein the metal consists essentially of copper, zinc, aluminum, beryllium, magnesium, lithium, or an alloy of copper, zinc, aluminum, beryllium, magnesium, lithium.

Item 48. The device according to item 47, wherein the metal consists essentially of copper or an alloy containing copper.

Item 49. The device according to item 45, wherein the non-metallic compound includes graphene, superconductors, semiconductors, or any combination thereof.

Item 50. The device according to any one of items 27 through 49, wherein the dielectric layer having a relative permittivity εᵣ and a thickness t_{d}, wherein the product of t_{d}*εᵣ is not greater than 500 microns, not greater than 400 microns, not greater than 300 microns, not greater than 250 microns, not greater than 200 microns, not greater than 150 microns, not greater than 100 microns, not greater than 90 microns, not greater than 80 microns, not greater than 70 microns, not greater than 60 microns, not greater than 50 microns, not greater than 45 microns, not greater than 40 microns, not greater than 38 microns, not greater than 36 microns, not greater than 34 microns, not greater than 32 microns, or not greater than 30 microns.

Item 51. A method to prepare material surfaces possessing anti-microbial properties using a semi-contiguous extreme low resistivity element in the form of a coating, film, foil, perforated foil or woven mesh that provides a highly conductive layer capable of disrupting in close proximity any electrical field produced by and for microbial species and where the extreme low resistivity element is physically and electrically isolated from the exterior surface by a layer of dielectric material.

Item 52. The method of item 51, wherein the extreme low resistivity element is silver.

Item 53. The method of item 51, wherein the extreme low resistivity element is copper.

Item 54. The method of item 51, wherein the extreme low resistivity element is gold.

Item 55. The method of item 51 wherein the extreme low resistivity element is aluminum.

Item 56. The method of item 51 wherein the extreme low resistivity element is beryllium.

Item 57. The method of item 51, wherein the primary dielectric element is opaque.

Item 58. The method of item 51 wherein the primary dielectric element is transparent or semi-transparent.

Item 59. A method to add anti-microbial properties to dressings and bandages using a semi-contiguous extreme low resistivity element in the form of a coating, film, foil, perforated foil or woven mesh that provides a highly conductive plane capable of disrupting in close proximity any electrical field produced by and for microbial species.

Item 60. The method of item 59 where a contiguous layer of dielectric film is affixed to the hyper-conductive element to prevent physical contact with tissue.

Item 61. The method of item 59 where typical fibers or materials used in dressings provide a dielectric physical barrier between the hyper-conductive element and tissue.

Item 62. A method to add anti-microbial properties to surgical sutures using a contiguous length of low resistivity element in the form of a wire incorporating a dielectric layer of insulation.

Item 63. A method to add anti-microbial properties to catheters by incorporating a flexible woven mesh of a hyper-conductive element embedded within the dielectric material of the catheter.

Item 64. A method to add anti-microbial properties to prostheses and medical implants using a semi-contiguous extreme low resistivity element in the form of a coating, film, foil, perforated foil or woven mesh that provides a highly conductive layer capable of disrupting in close proximity any electrical field produced by and for microbial species.

Item 65. The method of item 64 wherein the extreme low resistivity element is isolated from the tissue with a layer of dielectric material.

Item 66. The method of item 64 wherein the antimicrobial properties are added to prosthesis sockets or socket-liners.

Item 67. The method of item 64 wherein the anti-microbial properties are added to the surface of medical implants, and their respective fasteners, electrodes, wires or other components that serve as a surface which could otherwise harbor harmful microbes.

Item 68. A method to add anti-microbial properties to containers using a semi-contiguous extreme low resistivity element in the form of a coating, film, foil, perforated foil or woven mesh that provides a highly conductive layer capable of disrupting in close proximity any electrical field produced by and for microbial species.

Item 69. The method of item 68 wherein the extreme low resistivity element is isolated from the fluid by a layer of dielectric material.

Item 70. The method of item 68 wherein the antimicrobial properties are added to fluid containers.

### EXPERIMENTS

Microbe Field Disruption was tested using a modified US Environmental Protection Agency (“EPA”) Protocol on “Test Method for the Continuous Reduction of Bacterial Contamination on Copper Alloy Surfaces.” The test organism used was Enterobacter aerogenes.

Preparation of Samples

A polyester film (0.0005″) was laminated to a copper film (0.002″ of C10000) having an adhesive surface and cut into 1″×1″squares. Control squares of sole polyester film were prepared as well. Test and control squares were wiped clean with alcohol, rinsed with deionized water, air-dried and placed into a plastic Petri dish matted with two pieces of filter paper using sterile forceps.

Preparation of Test Organism

From stock cultures, Enterobacter aerogenes was inoculate in tubes of Tryptic Soy broth (TSB) and incubate for 24±2 hours at 22-25° C. Using a 4-mm inside diameter disposable sterile plastic transfer loop, three consecutive daily transfers of cultures in Tryptic Soy Broth were performed prior to use as inoculum followed by two (2) loopful transfers of culture into 10 ml broth medium. Cultures were thoroughly mixed on a “vortex” mixer and allow to settle. 0.09 mL of Triton X-100 was added. The upper two thirds of this suspension were decanted and used as the inoculum for testing.

Inoculation of Carriers

Test and control squares were inoculated all with 10 μl of culture using a calibrated pipette. The inoculum was spread within ⅛″ of the edges with a sterile spreader on the surface of the carriers. The carriers were allowed to dry at ambient conditions for the duration of the exposure. The exposure period begins with the initial inoculation of the carrier. Exposure was conducted over the course of 12 hours.

Harvesting Carriers.

Each test and control square was transferred into 30 mL neutralizing Letheen broth in sterile stars. All samples were sonicated in a waterbath for 18 to 22 seconds and shaken in an orbital shaker for 3 to 4 minutes at 250 RPM.

Preparation of Dilutions for Plating

Within 1 hour of neutralization, both supernatant Letheen broth of test and control samples were serially diluted. Control samples were diluted 1:100 (10⁻²); 1:1000 (10⁻³); and 1:10,000 (10⁻⁴) using deionized water. One test sample remained undiluted (10⁰) and other test samples were diluted, to 10⁻¹, 10⁻³, and 10⁻⁴. E. aerogenes source culture dilutions were prepared 10⁻², 10⁻³, and 10⁻⁴. 50 μl of each dilution was put on agar plates and incubated for 48 hours at 22-24 deg C.

Results

Colony-forming units were determined according to the Data Analyis of the EPA protocol. The number of organisms surviving per carrier were calculated:

CFU/carrier=((average number colonies/plate @ dilution)×(dilution factor)×(volume neutralized solution))/(volume plated)

Accordingly, culture source count were 4.2×10⁸/mL; control surface count=6.5×10⁷ CFU/mL and the test sample had no detectable colonies at any dilution.

The fact that not detectable colonies were formed means a log-7 reduction from a 12 hour exposure to a 0.0005″ dielecteric backed with copper without any direct contact between the microorganism and the copper surface. It follows that only the filed disruption accounts for the inactivation/no-growth outcome.

1. An antimicrobial device comprising:
a hyper-conductive layer; and
a dielectric layer adjacent to the hyper-conductive layer, wherein the antimicrobial device, has sufficient antimicrobial efficacy by a microbial field disruption effect.

2. The antimicrobial device according to claim 1 comprising an antimicrobial epidermal product or wound care product.

3. A molded component comprising:
a dielectric material having a non-planar surface, and
a hyper-conductive layer adjacent to the dielectric material.

4. The antimicrobial device according to claim 1, wherein the dielectric layer is in direct contact with the hyper-conductive layer.

5. The antimicrobial device according to claim 1, wherein the hyper-conductive layer includes a metal.

6. The antimicrobial device according to claim 5, wherein the metal includes copper, silver, gold, aluminum, beryllium, or any alloys containing copper, silver, gold, aluminum, beryllium.

7. The antimicrobial device according to claim 1, wherein the hyper-conductive layer is in a form of a solid foil, a perforated foil, film, a woven mesh, a non-woven mesh, or a grid.

8. (canceled)

9. The antimicrobial device according to claim 1, wherein the hyper-conductive layer has a thickness sufficient to form a conductive plane.

10. (canceled)

11. The antimicrobial device according to claim 9, wherein the thickness of the hyper-conductive layer is between 0.001″ and 0.005″.

12. The antimicrobial device according to claim 1, wherein the dielectric layer comprises a dielectric polymer.

13. The antimicrobial device according to claim 1, wherein the dielectric layer has a thickness sufficient to isolate the hyper-conductive layer from direct contact with a microbe.

14. The antimicrobial device according to claim 13, wherein the thickness of the dielectric layer is less than 0.001″.

15. (canceled)

16. The antimicrobial device according to claim 1, wherein the hyper-conductive layer or the dielectric layer can change shape without damaging the hyper-conductive layer or without damaging the dielectric layer.

17. The antimicrobial device according to claim 1, wherein the antimicrobial device, the antimicrobial epidermal product or wound care product, or the molded component has an antimicrobial efficacy within 3 mm of a surface of the hyper-conductive layer.

18. The antimicrobial device according to claim 3, further comprising a base layer overlying the hyper-conductive layer, the base layer located opposite to the dielectric layer.

19. (canceled)

20. The antimicrobial device according to claim 1, wherein the antimicrobial device according to any one of the preceding claims is an external medical article, an internal medical article, or a non-medical article.

21. The antimicrobial device according to claim 20, wherein the external medical article includes a bandage, gauze, compression, tube, triangular, dressings, absorptive, gauze, woven, filler, drape, cover, sterilization wraps, envelope, prosthetic sleeve, plasma bag, IV bag, IV tubing, IV fitting, communication device, button, cord, telephone, pain regulation patient pendant, probe, sphygmomanometer sleeve, sphygmomanometer cuff, tray, container, bed rail, chair, arm rest, or over-bed table surfaces.

22-50. (canceled)

51. A method to prepare material surfaces possessing anti-microbial properties using a semi-contiguous extreme low resistivity element in the form of a coating, film, foil, perforated foil or woven mesh that provides a highly conductive layer capable of disrupting in close proximity any electrical field produced by and for microbial species and where the extreme low resistivity element is physically and electrically isolated from the exterior surface by a layer of dielectric material.

52. (canceled)

53. The method of claim 51, wherein the extreme low resistivity element is copper.

54-57. (canceled)

58. The method of claim 51 wherein the primary dielectric element is transparent or semi-transparent.

59-70. (canceled)
